(19) United States
(12) Patent Application Publication
(10) Pub. No.: US 2025/0051205 A1
(43) Pub. Date: Feb. 13, 2025

(54) BIOGAS PRODUCTION DEVICE INCLUDING CONDUCTIVE SUPPORT-BASED DYNAMIC MODULE AND BIOGAS PRODUCTION METHOD USING SAME DEVICE

(71) Applicant: INDUSTRY-ACADEMIC COOPERATION FOUNDATION, YONSEI UNIVERSITY, Seoul (KR)

(72) Inventors: Sang-Hyoun KIM, Daegu (KR); Saint Moon KIM, Seoul (KR); Young-Bo SIM, Seoul (KR); Jisu YANG, Busan (KR)

(21) Appl. No.: 18/799,109

(22) Filed: Aug. 9, 2024

(30) Foreign Application Priority Data
Aug. 10, 2023 (KR) 10-2023-0105017

(51) Int. Cl. C02F 3/28; C02F 3/00; C12P 3/00

(52) U.S. Cl. CPC C02F 3/2806; C02F 3/005; C12P 3/00; C02F 2209/42; C02F 2301/046

(57) ABSTRACT

Embodiments include a biogas production device including a conductive support-based dynamic biofilm with improved biogas productivity by using, as a support, a conductive support having a surface on which a dynamic biofilm of an electroactive microbial community is formed, and a biogas production method using the device. The biogas production device comprises: a reaction tank for producing an organic wastewater mixture by mixing organic wastewater and anaerobic microbial community formation to produce biogas; a dynamic biofilm module for filtering the organic wastewater mixture transferred from the reaction tank using a porous conductive support having a dynamic biofilm formed on a surface thereof; and a storage tank for measuring and storing an amount of biogas produced in the reaction tank.

### CROSS-REFERENCE TO RELATED APPLICATIONS

This application claims the priority of Korean Patent Application No. 10-2023-0105017 filed on Aug. 10, 2023, in the Korean Intellectual Property Office, the disclosure of which is incorporated herein by reference.

### BACKGROUND

The disclosure relates to a biogas production device, and more specifically, to a biogas production device including a porous conductive support-based dynamic module and a method for producing biogas using the device.

As global interest in carbon neutrality and sustainable society increases, achieving sustainable energy goals in the near future is important. Although fossil fuels are still the main source of energy, there is a global movement to gradually transition to clean energy sources with low emissions. In this context, hydrogen is widely considered a promising energy carrier due to its environmentally sustainable properties, high energy density (122 J/g), and applicability to various applications. However, although many technologies have been developed in recent decades for this transition to a hydrogen economy, most hydrogen is produced from energy-intensive fossil fuels, and the production of hydrogen emits a lot of carbon, making the term “green hydrogen” meaningless.

To solve this problem, there is an increasing movement to produce biohydrogen. Biohydrogen is produced using living microorganisms, and there is a method of producing hydrogen by utilizing methane obtained by microorganisms decomposing organic matter consisting of carbon and hydrogen. This method can be said to be truly green hydrogen with no carbon emissions and carbon neutrality.

Therefore, it is becoming important to produce biohydrogen that can decarbonize the energy system and valorize low-value wastes by utilizing biomass, and for this reason, technologies for producing hydrogen from a wide range of organic wastes, including algae biomass, food waste, and agricultural waste, are being researched and developed.

However, despite these efforts, there are still problems in the production of biohydrogen using biomass, such as low productivity due to various impurities, and lower efficiency compared to hydrogen production using fossil fuels.

### SUMMARY

An aspect of the disclosure is to provide a biogas production device including a dynamic module based on a conductive support having a dynamic biofilm formed on the surface thereof.

Another aspect of the disclosure is to provide a method for producing biogas using a dynamic module based on a conductive support having a dynamic biofilm formed on the surface thereof.

The aspect of the disclosure is not limited to that mentioned above, and other aspects not mentioned will be clearly understood by those skilled in the art from the description below.

An example of the disclosure provides a biogas production device including a conductive support-based dynamic module.

According to an example of the disclosure, the device may include a conductive support-based dynamic module including: a reaction tank for producing an organic wastewater mixture by mixing organic wastewater and anaerobic microbial community formation to produce biogas; a dynamic biofilm module for filtering the organic wastewater mixture transferred from the reaction tank using a porous conductive support having a dynamic biofilm formed on the surface thereof; and a storage tank for measuring and storing the amount of biogas produced in the reaction tank.

In addition, according to an example of the disclosure, the device may further include a circulation pump that transfers the organic wastewater mixture from the reaction tank to the dynamic biofilm module.

In addition, according to an example of the disclosure, the device may further include a return pipe that returns the organic wastewater mixture and a solid filtered and separated from the dynamic biofilm module to the reaction tank.

In addition, according to an example of the disclosure, the device may further include inside the reaction tank, a level controller that controls the level of the organic wastewater mixture, and a stirrer that creates the organic wastewater mixture.

In addition, according to an example of the disclosure, the porous conductive support may be composed of an acid-treated conductive mesh.

In addition, according to an example of the disclosure, the porous conductive support may include at least one selected from the group consisting of stainless steel, carbon fiber, and carbon nanotube.

In addition, according to an example of the disclosure, the pore size of the porous conductive support may be 100 μm to 444 μm.

In addition, according to an example of the disclosure, in order to discharge filtered treated water after filtering the organic wastewater mixture in the dynamic biofilm module, an effluent pump may be coupled to the dynamic biofilm module.

Another example of the disclosure provides a method for producing biogas using a conductive support-based dynamic module.

In addition, according to an example of the disclosure, the method may include: an injection step of injecting organic wastewater and anaerobic microbial community formation into the reaction tank; a reaction step of generating biogas by making the organic wastewater and anaerobic microbial community formation into an organic wastewater mixture using a stirrer in the reaction tank; a filtration step of filtering the organic wastewater mixture transferred from the reaction tank through a conductive support so that a dynamic biofilm is formed on the surface of the conductive support and a solid filtered and separated is generated; a return and discharge step of returning the organic wastewater mixture and the solid filtered and separated in the filtration step to the reaction tank and discharging the filtered treated water; and a storage step of storing the biogas generated in the reaction step in a storage tank and measuring the amount of the biogas.

In addition, according to an example of the disclosure, the filtered and separated solid may be a biomass containing an electroactive microbial community.

According to an example of the disclosure, a biogas production device including a conductive support-based dynamic module provides an effect of enhancing hydrogen production efficiency compared to a biogas production device using a non-conductive support.

According to an example of the disclosure, a biogas production device including a conductive support-based dynamic module provides an effect of having a fixed shape and being easy to operate for a long period of time by using a conductive material as a support.

The effects of the disclosure are not limited to the effects described above, and should be understood to include all effects that are inferable from the configuration of the disclosure described in the detailed description or claims of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a configuration diagram of a biogas production device including a conductive support-based dynamic module according to an example of the disclosure;

FIGS. 2A, 2B, and 2C are each a graph showing hydrogen productivity according to the injection of a conductive material;

FIG. 3 is a graph showing the ratio of homoacetogenesis and H2 producing acetate metabolic pathways according to the injection of a conductive material;

FIGS. 4A and 4B are each a graph comparing hydrogen productivity according to the type of a porous support;

FIGS. 5A and 5B are each a graph comparing microbial communities in a biofilm process, generated according to the type of a porous support; and

FIG. 6 is a flow chart showing a biogas production method using a conductive support-based dynamic module according to an example of the disclosure.

### DETAILED DESCRIPTION

Hereinafter, the disclosure will be described with reference to the accompanying drawings.

However, the disclosure may be implemented in various different forms and, therefore, is not limited to the examples described herein. In order to clearly explain the disclosure in the drawings, portions unrelated to the description are omitted, and similar portions are given similar reference numerals throughout the specification.

Throughout the specification, when a portion is said to be “connected (linked, contacted, combined)” with another portion, this includes not only a case of being “directly connected” but also a case of being “indirectly connected” with another member in between. In addition, when a portion is said to “include” a certain component, this does not mean that other components are excluded, but that other components may be added, unless specifically stated to the contrary.

The terms used herein are merely used to describe specific examples and are not intended to limit the disclosure. Singular expressions include plural expressions unless the context clearly dictates otherwise. In this specification, it should be understood terms such as “include” or “have” are to designate the presence of features, numbers, steps, operations, components, parts, or combinations thereof described in the specification, but are not to exclude in advance the possibility of the presence or addition of one or more other features, numbers, steps, operations, components, parts, or combinations thereof.

Hereinafter, examples of the disclosure will be described in detail with reference to the accompanying drawings.

A biogas production device including a conductive support-based dynamic module according to an example of the disclosure will be described.

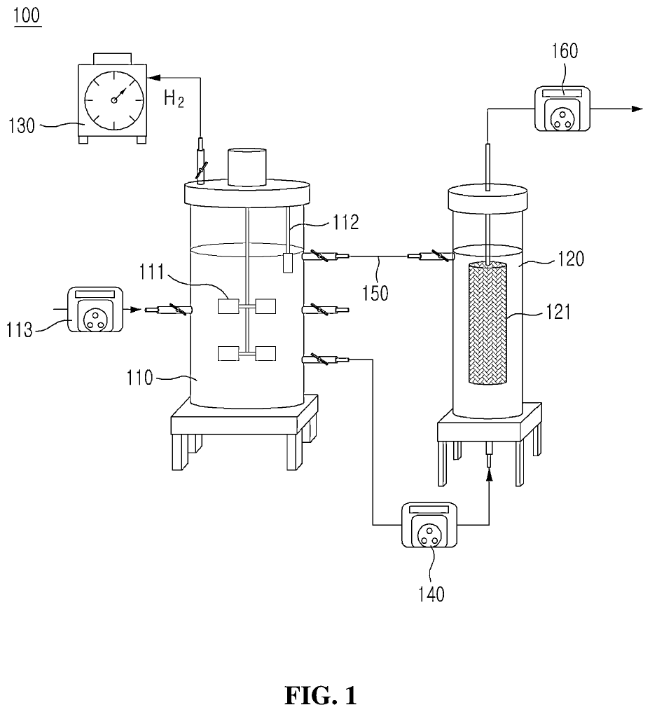

FIG. 1 is a configuration diagram of a biogas production device including a conductive support-based dynamic module according to an example of the disclosure.

Referring to FIG. 1, a biogas production device 100 including a conductive support-based dynamic module according to an example of the disclosure may include: a reaction tank 110 for producing an organic wastewater mixture by mixing organic wastewater and anaerobic microbial community formation to produce biogas; a dynamic biofilm module 120 for filtering the organic wastewater mixture transferred from the reaction tank using a porous conductive support 121 having a dynamic biofilm formed on the surface thereof; and a storage tank 130 for measuring and storing the amount of biogas produced in the reaction tank.

At this time, in the reaction tank 110, organic wastewater and anaerobic microbial community formation are mixed to form an organic wastewater mixture, and the organic wastewater mixture undergoes an anaerobic digestion process through anaerobic microorganisms to generate biogas. The biogas generated at this time may include methane or hydrogen, and the preferred biogas is hydrogen.

Meanwhile, a level controller 112 for controlling the water level of the organic wastewater mixture and a stirrer 111 for creating the organic wastewater mixture may exist inside the reaction tank 110.

At this time, the level controller 112 is connected to an effluent pump 160, so that the amount of treated water to be discharged through the level controller 112 is controlled while constantly controlling the water level of the reaction tank 110.

In addition, the feedstock such as a substrate, organic wastewater, and anaerobic microbial community formation injected into the stirrer 111 may be stirred to continuously perform the reaction.

In addition, the reaction tank 110 may be a continuous stirring tank reactor that continuously maintains the reaction while constantly controlling the water level through the level controller 112.

Meanwhile, the biogas production device 100 including a conductive support-based dynamic module may further include a circulation pump 140 that transfers the organic wastewater mixture from the reaction tank 110 to the dynamic membrane module 120.

At this time, the reaction speed may be controlled by controlling the flow rate of the organic wastewater mixture to be transferred to the dynamic membrane module 120 using the circulation pump 140.

Meanwhile, the dynamic membrane module 120 may include a porous conductive support 121 having a surface on which a dynamic biofilm is formed.

At this time, the porous conductive support may be composed of an acid-treated conductive mesh.

At this time, the porous conductive support may form a dynamic biofilm on the surface, and the dynamic biofilm may be formed when the organic wastewater mixture passes through the porous support in the dynamic membrane module 120.

At this time, the biofilm formed is an anaerobic microbial community, and various types of anaerobic microbial communities may be formed, wherein since the porous conductive support has conductive properties, a microbial community composed mainly of electroactive microorganisms may be formed.

The electroactive microorganism may perform electron emission activity enabling to transport electrons across a biofilm, and if the electron transfer is improved, it is possible to overcome the thermodynamic and kinetic limitations of dark fermentation hydrogen production, thereby improving the production of biogas.

In addition, in the case of dynamic biofilms composed of electroactive microorganisms, electrons generated from reduced paradoxin may be transferred to hydrogenases of adjacent electroactive H2 producing bacteria through a porous conductive support and an electroactive extracellular polymeric substance (EPS) through a cell membrane, and this electron transfer may share H2 production capacity while preventing H2 consumption pathways.

In addition, the porous conductive support may serve as a physical support for microbial growth and stimulate molecules of extracellular substances such as enzymes, microbial nutrients, and electron shuttles.

These results are ultimately related to the change in metabolic flow toward a hydrogen production acetogenesis pathway, and the high NADH/NAD+ ratio and low redox potential shown in a conductive support-based continuous reactor may provide a favorable environment for reduction, thereby promoting the NHDH-based hydrogen production pathway, enabling additional hydrogen production. Through this mechanism, the productivity of hydrogen, which is biogas, may be improved.

In addition, another reason for the improvement in biogas productivity is that the average particle size of the electroactive microbial community generated by the porous conductive support increases, and the electroactive microbial community generated by the porous conductive support is granulated larger than the size of the conventionally generated microbial community, and thus the particles are larger than the size of the pores of the porous conductive support, so that the electroactive microbial community generated is not lost through the pores of the support, and an effect of enhancing the productivity of biogas may be secured.

In addition, since the porous conductive support has a fixed shape, even if a conductive material is not continuously injected, the concentration of the conductive material may be maintained above a certain concentration, which allows application thereof to a continuous reactor.

Therefore, the biogas production device including a conductive support-based dynamic module according to an example of the disclosure may include a continuous reactor.

Accordingly, a reaction for biogas production may be performed continuously and persistently for a long period of time, which may further improve the productivity of hydrogen, which is biogas.

Meanwhile, the pore size of the porous conductive support may be 100 μm to 444 μm.

At this time, if the pore size of the porous conductive support is less than 100 μm, membrane fouling may occur as the biomass particles in the reaction tank are granulated, and if the size exceeds 444 μm, there may be a problem of excessive loss of microorganisms before the biomass granulation at the beginning of the reaction tank operation.

In addition, the acid-treated porous conductive support may include at least one selected from the group consisting of stainless steel, carbon fiber, and carbon nanotube.

At this time, the reason for performing the acid treatment is to remove contaminants on the surface of the porous conductive support, and the acid treatment process may remove nitrogen functional groups including pyrrole nitrogen and pyridine nitrogen that interfere with electron transfer between microorganisms and conductive materials.

Meanwhile, the biogas production device 100 including a conductive support-based dynamic module may further include a return pipe 150 that returns an organic wastewater mixture and a solid filtered and separated from the dynamic membrane module 120 to the reaction tank 110.

At this time, the return pipe 150 may be controlled according to the level of the organic wastewater and the amount of the solid filtered and separated, for the return to the reaction tank 110.

Meanwhile, an effluent pump 160 may be coupled to the dynamic membrane module 120 to discharge the filtered treated water after filtering the organic wastewater mixture.

At this time, the filtered treated water may contain hydrogen generated from an anaerobic microbial community, and a separation tank may be additionally coupled to the effluent pump 160 to separate the hydrogen from the treated water.

A biogas production method using a conductive support-based dynamic module according to another example of the disclosure will be described.

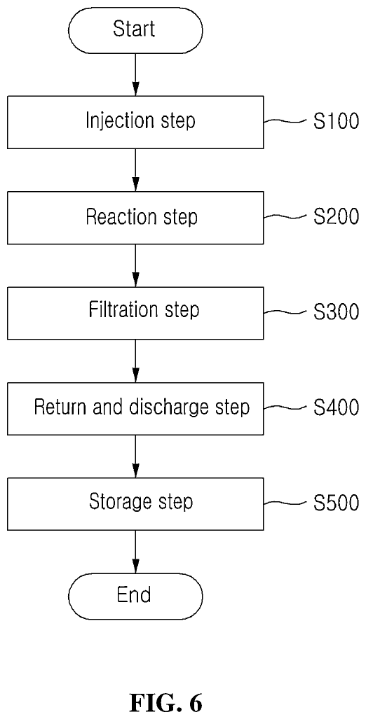

FIG. 6 is a flow chart showing a biogas production method using a conductive support-based dynamic module according to an example of the disclosure.

Referring to FIG. 6, a biogas production method using a conductive support-based dynamic module may include: an injection step S100 of injecting organic wastewater and anaerobic microbial community formation into the reaction tank; a reaction step S200 of generating biogas by making the organic wastewater and anaerobic microbial community formation into an organic wastewater mixture using a stirrer in the reaction tank; a filtration step S300 of filtering the organic wastewater mixture transferred from the reaction tank through a conductive support so that a dynamic biofilm is formed on the surface of the conductive support and a solid filtered and separated is generated; a return and discharge step S400 of returning the organic wastewater mixture and the solid filtered and separated in the filtration step to the reaction tank and discharging the filtered treated water; and a storage step S500 of storing the biogas generated in the reaction step in a storage tank and measuring the amount of the biogas.

A first step may be an injection step of injecting organic wastewater and anaerobic microbial community formation into the reaction tank. (S100)

At this time, anaerobic granular sludge may be used as the anaerobic microbial community formation material.

Therefore, a feedstock including anaerobic granular sludge, organic wastewater, and a substrate for microbial community formation may be injected into the reaction tank, and the raw material may be stirred and reacted to generate an organic wastewater mixture. At this time, as for the anaerobic granular sludge and the organic wastewater, anything that can produce biogas is possible without limitation.

In addition, the anaerobic granular sludge may be heat-treated at 70° C. to 110° C. for 10 to 50 minutes before being injected into the reaction tank, to remove methane-producing substances and enrich bacteria that produce H2.

In addition, the substrate may include, for example, glucose.

In addition, the feedstock may further include supplements such as NaHCO₃, NH4HCO3, FeSO47H2O, CuSO45H2O, MgCl26H2O, CoCl25H2O, KH2PO4, and MnSO46H2O.

A second step may be a reaction step of generating biogas by making the organic wastewater and anaerobic microbial community formation into an organic wastewater mixture using a stirrer in the reaction tank. (S200)

At this time, the biogas generated may contain methane and hydrogen. Preferably, hydrogen may be contained.

A third step may be a filtration step of filtering the organic wastewater mixture transferred from the reaction tank through a conductive support so that a dynamic biofilm is formed on the surface of the conductive support and a solid filtered and separated is generated. (S300)

At this time, in the case of the filtration step in which the conductive support is filtered, it is recommended to maintain the hydraulic retention time (HRT) at 12 hours to 2 hours.

At this time, if the hydraulic retention time is less than 2 hours, there may be a problem such as a loss of attached and floating microorganisms or a decrease in the substrate consumption rate in the reaction tank, and it is most effective to perform operations under the condition of the hydraulic retention time of 12 hours at the beginning of the operation.

A fourth step may be a return and discharge step of returning the organic wastewater mixture and the solid filtered and separated in the filtration step to the reaction tank and discharging the filtered treated water. (S400)

At this time, the amount returned to the reaction tank may be adjusted according to the amount of the filtered and separated solid and organic wastewater mixture.

In addition, the filtered and separated solid may be returned to the reaction tank, and the organic wastewater mixture may be subjected to anaerobically reacting in the reaction tank to produce a large amount of biogas.

A fifth step may be a storage step of storing the biogas generated in the reaction step in a storage tank and measuring the amount of the biogas. (S500)

At this time, a gas flow meter may be additionally included in the storage tank to measure the amount of the biogas.

In addition, the stored biogas may be gas mixed with methane, hydrogen, etc., and thus a separation process step of separating the gas may be additionally performed. At this time, the separation process step may be performed using a conventional method.

Hereinafter, the disclosure will be described in more detail through examples. These examples are only intended to illustrate the disclosure, and the scope of the disclosure is not limited by these examples.

### Example 1

Anaerobic granular sludge (Cheongju, Korea) collected from an anaerobic sludge blanket reactor (UASB) for brewery wastewater treatment was used as an anaerobic microbial community formation. The characteristics of the anaerobic granular sludge were total solids (TS) 70.8 g/L, volatile solids (VS) 68.3 g/L, and pH 7.5. The anaerobic granular sludge was heat-treated at 90° C. for 30 min in a tank before being injected into the device to destroy methanogens and enrich H2 producing bacteria. Glucose was used as a sole substrate at a concentration of 20.0 g/L, and the feedstock was supplemented with NaHCO₃ (7.0 [symbol] 10.0 g/L), NH4HCO3 0.3 g/L, FeSO47H2O 0.025 g/L, CuSO45H2O 0.005 g/L, MgCl26H2O 0.1 g/L, CoCl25H2O 0.0001 g/L, KH2PO4 0.125 g/L, and MnSO46H2O 0.015 g/L.

A biogas production device (DMBR) composed of a continuous stirring tank reactor (CSTR) with a sidestream porous conductive dynamic membrane (DM) module was used. The individual working volumes of the CSTR and DM module were 2 L, and a stirrer was installed in the CSTR to homogenize the injected material at 150 rpm.

In addition, a stainless-steel mesh (444 μm pore size) treated with formic acid was used as the porous conductive support, and formic acid pretreatment was performed to remove contaminants on the surface of the stainless-steel mesh.

The fluid was recirculated through a peristaltic pump between the CSTR and the DM module, 10% (v/v) (0.4 L) of anaerobic microbial culture was injected, and the remainder was supplied together with the feedstock.

Then, pure N2 gas was injected into the DMBR to maintain strict anaerobic conditions in the system. After maintaining a batch mode for 13 h, the DMBR was operated in a continuous mode.

The feedstock was continuously supplied to the CSTR in the continuous mode, and a discharge pump attached to a water level sensor was maintained to discharge a constant amount. The device was operated with the hydraulic retention time (HRT) reduced from 8 h to 4 h, and finally to 2 h.

### Example 2

The same process as in example 1 was carried out, but a carbon fiber mesh was used as the porous support of the DM module.

### Comparative Example

The same process as in Example 1 was carried out, but a polyester mesh was used as the porous support of the DM module.

### Experimental Example

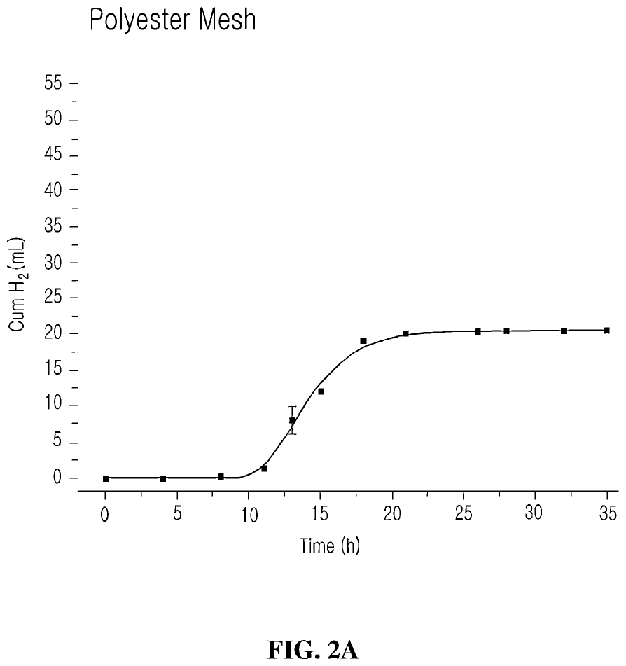

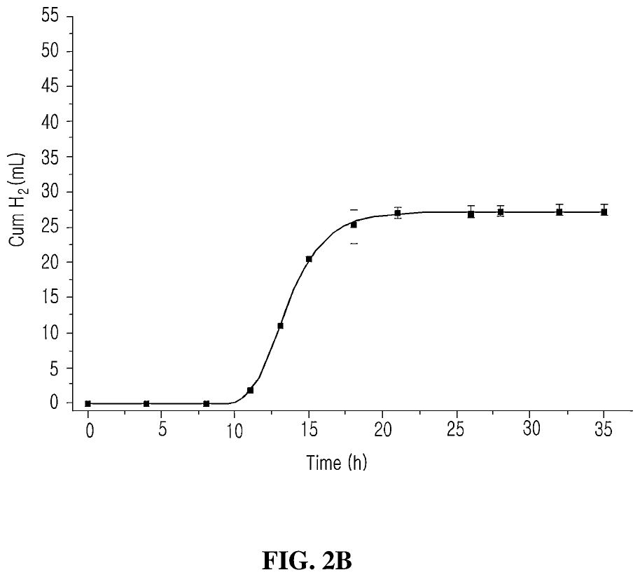

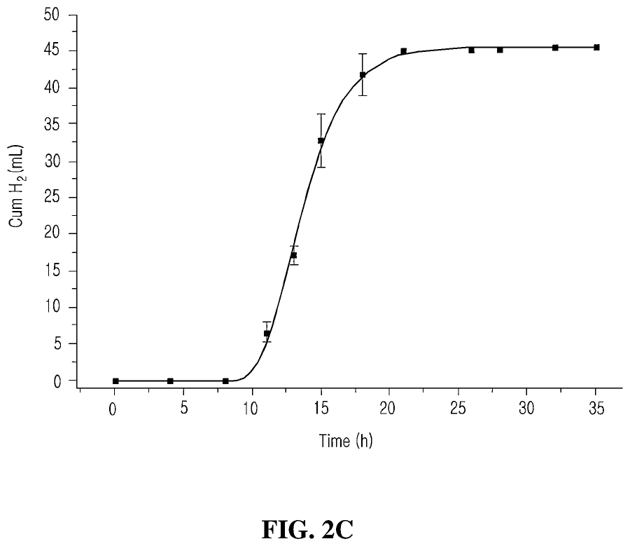

FIGS. 2A, 2B, and 2C are each a graph showing hydrogen productivity according to conductive material injection.

Referring to FIGS. 2A, 2B, and 2C, it is possible to confirm that compared to when injecting polyester, a non-conductive material (FIG. 2A), hydrogen productivity is improved by 78.9% when injecting a carbon fiber mesh, a conductive material (FIG. 2B), and by 125.4% when injecting a stainless-steel mesh (FIG. 2C).

Accordingly, it is possible to know that the conductive material is involved in enhancing hydrogen productivity.

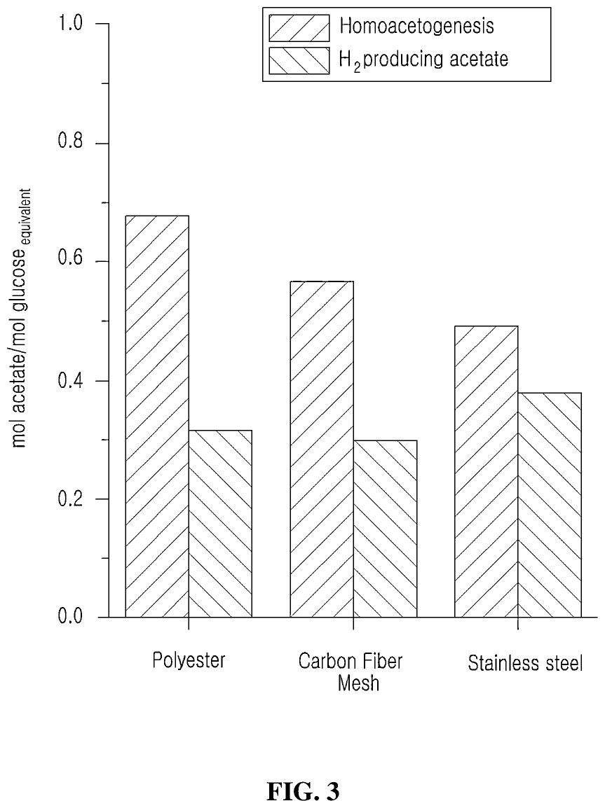

FIG. 3 is a graph showing the ratio of homoacetogenesis and H2 producing acetate metabolic pathways according to the injection of a conductive material.

Referring to FIG. 3, it is possible to know that compared to when injecting polyester, a non-conductive material, the homoacetogenesis metabolic pathway that consumes hydrogen is reduced when injecting a carbon fiber mesh, and the homoacetogenesis metabolic pathway that consumes hydrogen is reduced and the H2 producing acetate metabolic pathway that produces hydrogen is increased when injecting stainless steel.

Accordingly, it is possible to know that when a conductive material is injected, a Homoacetogenesis metabolic pathway that produces hydrogen decreases and an H2 producing acetate metabolic pathway that produces hydrogen increases, thereby enhancing hydrogen productivity.

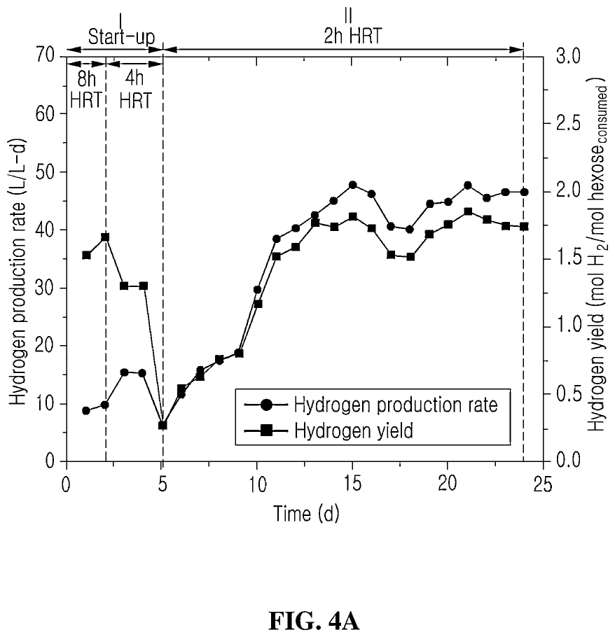

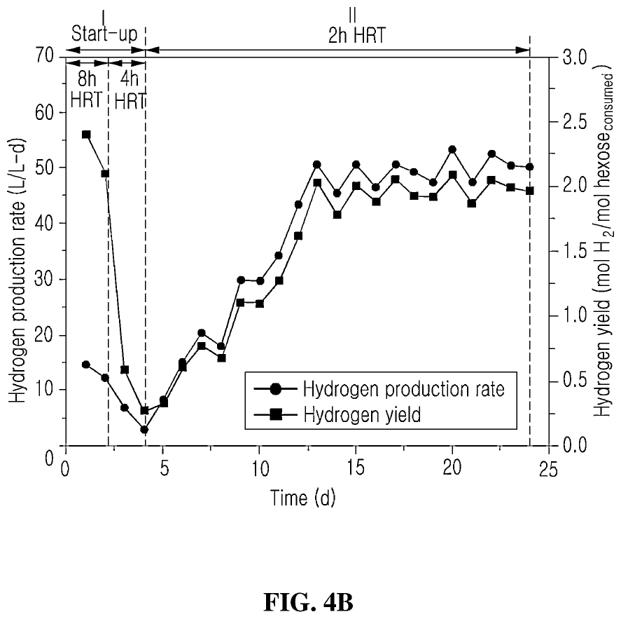

FIGS. 4A and 4B are each a graph comparing hydrogen productivity according to the type of a porous support.

Referring to FIGS. 4A and 4B, shown are the results of an experiment conducted with different types of porous supports in a biogas production device according to an example of the disclosure, wherein FIG. 4A is when a non-conductive polyester mesh produced in a comparative example is used as a porous support, and FIG. 4B is when a conductive stainless-steel produced in example 1 is used as a porous support. In FIGS. 4A and 4B, the experiment was conducted in a continuous process.

At this time, referring to the graphs, it is possible to confirm that the hydrogen productivity was improved by 16.8% and the hydrogen yield was improved by 20.4% when stainless steel was used as a conductive support (FIG. 4B).

As shown in Table 1 below, in a biogas production device including a conductive support-based dynamic module according to an example of the disclosure, the time when a polyester mesh was used as a support is shown as process I, the time when stainless steel was used as a support (example 1) is shown as process II, and metabolic pathways according to the respective supports were compared.

Referring to Table 1 below, it is possible to confirm that in process II, which uses stainless steel as a support, the lactic acid pathway and homoacetogenesis pathway that consume hydrogen were reduced, and the acetogenesis pathway that produces hydrogen was strengthened compared to process I, which uses a polyester mesh as a support.

TABLE 1

| | | | Hydrogen |
|---|---|---|---|
| | | | production |
| | | Homoacetogenesis | Acetogenesis |
| | Lactate pathway | Pathway (mol | pathway(mol |
| | (mol lactate/mol | acetate/mol | acetate/mol |
| | hexose_{consumed}) | hexose_{consumed}) | hexose_{consumed}) |
| Process I | 0.14 ± 0.01 | 0.15 ± 0.01 | 0.40 ± 0.02 |
| (polyester) | | | |
| Process II | 0.11 ± 0.01 | 0.05 ± 0.01 | 0.50 ± 0.02 |
| (stainless-steel) | | | |

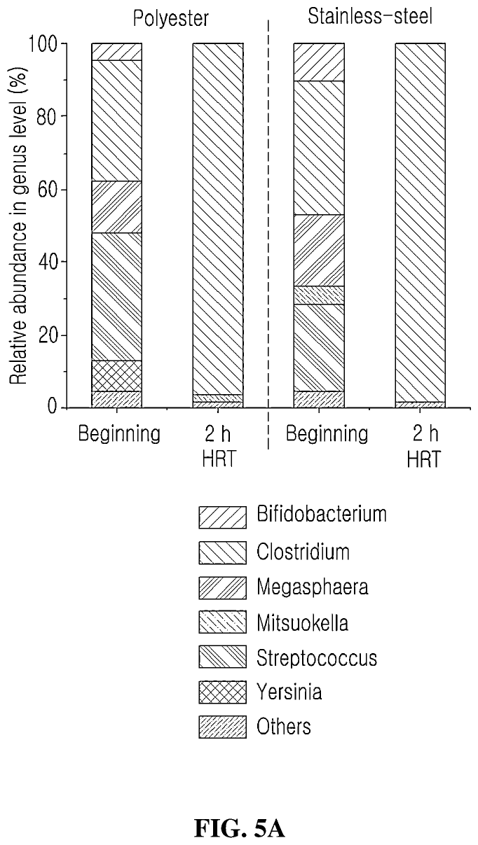

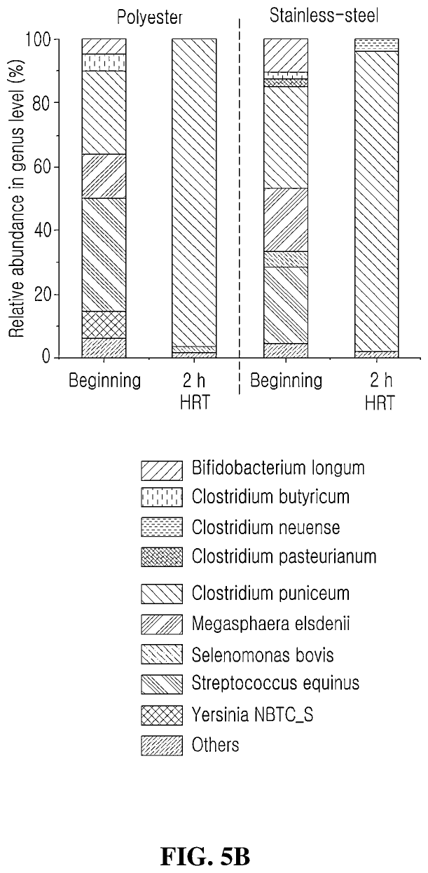

FIGS. 5A and 5B are each a graph comparing microbial communities in a biofilm process, generated according to the type of a porous support.

Referring to FIGS. 5A and 5B, shown are the results of comparing the microbial communities generated when a non-conductive polyester mesh was used as a support (FIG. 5A) and when a conductive stainless-steel mesh was used as a support (FIG. 5B), and it is possible to confirm that both FIGS. 5A and 5B show similar communities at the beginning of the operation.

However, under the hydraulic retention time condition of 2 hours, where hydrogen productivity was the highest, different Clostridium species were found to be dominant, and it is possible to know that a non-electroactive microbial community was dominant when a polyester mesh was used as a support (FIG. 5A), and an electroactive microbial community was dominant when a stainless-steel mesh was used as a support (FIG. 5B).

Through the experimental examples, it is possible to know that when a porous conductive support is used, a biofilm dominated by an electroactive microbial community is formed, which significantly enhance the productivity of hydrogen, which is biogas.

In particular, it was confirmed that the hydrogen reaction efficiency was enhanced by producing biomass containing electroactive microbial communities with large particle sizes even in a short hydraulic retention time (HRT) of 2 hours, and the loss of microbial communities was reduced, further enhancing hydrogen productivity, and it was found that it was effective to form electroactive microbial communities using porous conductive support because electroactive microorganisms proceed with the reaction through a metabolic pathway efficient for hydrogen production, thereby improving the productivity of biohydrogen.

The description of the disclosure described above is for illustrative purposes, and those skilled in the art will understand that the disclosure is easily modifiable into other specific forms without changing the technical idea or essential features of the disclosure. Therefore, the examples described above should be understood in all respects as illustrative and not restrictive. For example, each component described as single may be implemented in a distributed manner, and similarly, components described as distributed may also be implemented in a combined form.

The scope of the disclosure is indicated by the claims described below, and all changes or modified forms derived from the meaning and scope of the claims and their equivalent concepts should be construed as being included in the scope of the disclosure.

### EXPLANATION OF REFERENCE NUMERALS

100: biogas production device including conductive support-based dynamic module
110: reaction tank
111: stirrer
112: level controller
113: injection port
120: dynamic membrane module
121: porous conductive support
130: storage tank
140: transfer pump
150: return pipe
160: effluent pump

What is claimed is:

1. A biogas production device including a conductive support-based dynamic biofilm, the biogas production device comprising:
a reaction tank for producing an organic wastewater mixture by mixing organic wastewater and anaerobic microbial community formation to produce biogas;
a dynamic biofilm module for filtering the organic wastewater mixture transferred from the reaction tank using a porous conductive support having a dynamic biofilm formed on a surface thereof; and
a storage tank for measuring and storing an amount of biogas produced in the reaction tank.

2. The biogas production device of claim 1, further comprising a circulation pump configured to transfer the organic wastewater mixture from the reaction tank to the dynamic biofilm module.

3. The biogas production device of claim 1, further comprising a return pipe configured to return the organic wastewater mixture and a solid filtered and separated from the dynamic biofilm module to the reaction tank.

4. The biogas production device of claim 1, further comprising a level controller configured to control a level of the organic wastewater mixture, and a stirrer configured to create the organic wastewater mixture, wherein the level controller and the stirrer are disposed inside the reaction tank.

5. The biogas production device of claim 1, wherein the porous conductive support is composed of an acid-treated conductive mesh.

6. The biogas production device of claim 1, wherein the porous conductive support comprises at least one selected from a group consisting of stainless steel, carbon fiber, and carbon nanotube.

7. The biogas production device of claim 1, wherein a pore size of the porous conductive support is 100 um to 444 um.

8. The biogas production device of claim 1, further comprising an effluent pump coupled to the dynamic biofilm module to discharge filtered treated water after filtering the organic wastewater mixture in the dynamic biofilm module.

9. A method for producing biogas using the biogas production device including the conductive support-based dynamic biofilm according to claim 1, the method comprising:
an injection step of injecting organic wastewater and anaerobic microbial community formation into the reaction tank;
a reaction step of generating biogas by mixing the organic wastewater and anaerobic microbial community formation to make an organic wastewater mixture using a stirrer in the reaction tank;
a filtration step of filtering the organic wastewater mixture transferred from the reaction tank through a conductive support so that a dynamic biofilm is formed on the surface of the conductive support and a solid filtered and separated is generated;
a return and discharge step of returning the organic wastewater mixture and the solid filtered and separated in the filtration step to the reaction tank and discharging filtered treated water; and
a storage step of storing the biogas generated in the reaction step in the storage tank and measuring an amount of the biogas.

10. The method of claim 9, wherein the filtered and separated solid is a biomass containing an electroactive microbial community.
